(19) United States
(12) Patent Application Publication
(10) Pub. No.: US 2026/0224410 A1
(43) Pub. Date: Aug. 6, 2026

(54) REUSABLE ARTICLES AND GARMENTS FOR RETAINING BODILY FLUIDS AND SOLIDS

(71) Applicant: MAS Innovation (Private) Limited, Battaramulla (LK)

(72) Inventors: Wijesinghe Mudiyanselage Shashika Virajinie Wijesinghe, Battaramulla (LK); Dharini Alexandra Speldewinde, Battaramulla (LK); Udara Pulajith Weeramunda Sayakkara, Battaramulla (LK); Kahandawala Arachchige Don Laksha Sandaruwan, Battaramulla (LK)

(73) Assignee: MAS Innovation (Private) Limited, Battaramulla (LK)

(21) Appl. No.: 19/146,140

(22) PCT Filed: Jan. 4, 2024
(86) PCT No.: PCT/SG2024/050007

§ 371 Date: Jul. 7, 2025

(30) Foreign Application Priority Data
Jan. 6, 2023 (SG) 10202300049U

(51) Int. Cl. A61F 13/15; A61F 13/42; A61F 13/511; A61F 13/512

(52) U.S. Cl. CPC A61F 13/15268; A61F 13/15203; A61F 13/42; A61F 13/51121; A61F 13/512; A61F 2013/15276

(57) ABSTRACT

Described is a reusable article for retaining bodily fluids and solids and a reusable garment including the reusable article. The reusable article includes a liquid impermeable inner layer, which has a first surface and a second surface, and a liquid impermeable barrier layer. The first surface is configured to face towards the skin of a user when in use. The liquid impermeable barrier layer and the liquid impermeable inner layer are attached to each other around their peripheries, forming a liquid retention chamber between the second surface of the liquid impermeable inner layer and the liquid impermeable outer layer. The liquid impermeable inner layer includes holes to allow a liquid to pass through the liquid impermeable inner layer into the liquid retention chamber.

### FIELD OF THE INVENTION

The present invention provides a reusable article for retaining bodily fluids and solids. The present invention also provides a reusable garment comprising the reusable article.

### BACKGROUND

The listing or discussion of any apparently prior-published document in this specification should not necessarily be taken as an acknowledgement that the document is part of the state of the art or is common general knowledge.

Conventional reusable articles for retaining bodily fluids and solids, such as diaper liners, sanitary pads, panty liners, and the like, typically comprise an absorbent core layer such as a terry towel layer and a leak-resistant outer cover made from a heavy polymer film. They are typically used as an absorbent component in garments that resemble underwear, such as diapers (e.g. training pants, pull-on pant-type diapers, refastenable diapers, pant-type diapers), incontinence briefs, or feminine hygiene undergarments, and the like. To increase liquid absorbency of such articles it is often necessary to increase the size of the absorbent area or add additional absorbent core units (layering), which can result in a bulky article that can be uncomfortable for the wearer. Reusable articles that have a limited liquid absorbance capacity can leak and expose the user's skin to bodily fluids and moisture, which can cause discomfort and skin irritation.

Thus, there remains a need for alternative and/or improved reusable articles for retaining bodily fluids and solids.

### SUMMARY

The present invention provides a reusable article for retaining bodily fluids and solids, the reusable article comprising:
a liquid impermeable inner layer having a first surface and a second surface, the first surface configured to face towards the skin of a user when in use; and
a liquid impermeable outer layer;
the liquid impermeable outer layer and the liquid impermeable inner layer being attached to each other around their peripheries, thereby forming a liquid retention chamber between the second surface of the liquid impermeable inner layer and the liquid impermeable outer layer,
the liquid impermeable inner layer comprising a plurality of holes to allow a liquid to pass through the liquid impermeable inner layer into the liquid retention chamber.

The present invention also provides a reusable garment comprising a reusable article of the present invention. In certain examples disclosed herein, the reusable garment is a reusable diaper, such as a training pant, a pull-on pant-type diaper, a refastenable diapers, or a pant-type diaper.

The inventors have found that the reusable articles of the present invention can collect and retain bodily fluids discharged by a user in a position that is away from the user's skin. Specifically, bodily fluids are collected and retained within the liquid retention chamber of the reusable article, which reduces the exposure of the user's skin to the bodily fluids, thus increasing comfort for the user once the article is soiled and/or reducing any skin irritation that may be cause by extended contact of the user's skin to the bodily fluids. In certain examples disclosed herein, the reusable article further comprises a liquid impermeable overlayer that extends over a portion or substantially all of the first surface of the liquid impermeable inner layer. The liquid impermeable overlayer comprises a plurality of recesses. The present inventors have found that the recesses of the liquid impermeable overlayer can act as receptacles for solid exudates discharged from a user, which can reduce the exposure to the user's skin to the solid exudates, thus further increasing comfort for a user once the reusable article is soiled and/or reduce any skin irritation that may be cause by the solid exudates. The recesses can also facilitate the passage of bodily fluids into the liquid retention chamber away from a user's skin.

Preferred but optional features are set out in the dependent claims. Additional aspects and embodiments of the reusable article and reusable garment of the present invention will be apparent from the following description, drawings and claims. As can be appreciated from the foregoing and following description, each and every feature described herein, and each and every combination of two or more such features, is included within the scope of the present disclosure provided that the features included in such a combination are not mutually inconsistent. In addition, any feature or combination of features may be specifically excluded from any embodiment.

### BRIEF DESCRIPTION OF THE DRAWINGS

FIG. 1 shows a partially exploded cross-sectional view of an exemplary reusable article of the present invention.

FIG. 2 shows a partially exploded cross-sectional view of the exemplary reusable article shown in FIG. 1 further comprising a liquid impermeable overlayer.

FIG. 3 shows a partially exploded cross-sectional view of the exemplary reusable article shown in FIG. 2, wherein the liquid retention chamber houses a liquid collection vessel rather than a liquid absorbent material.

FIG. 4 shows an isolated top view of the exemplary reusable article shown in FIG. 1 to FIG. 3. In this figure, the flange portions and any liquid impermeable inner layer that may extend past the peripheries of the liquid impermeable overlayer are not shown.

FIG. 5 shows a magnified view of a single recess and hole present in the exemplary reusable article shown in FIG. 1 to FIG. 4.

FIG. 6 shows a top view of a liquid absorbent unit which may be contained within the liquid retention chamber of the reusable article of the present invention.

FIG. 7 shows a top view of a liquid impermeable overlayer together with an isolated cross-sectional image of one of the recesses present in the liquid impermeable overlayer.

FIG. 8 shows an alternative liquid impermeable overlayer wherein the recesses of the liquid impermeable overlayer are in the form of vents.

FIG. 9 shows a further alternative liquid impermeable overlayer wherein the liquid impermeable overlayer has a waffle-like structure, with each recess having a rectangle based frustoconical shape.

FIG. 10A and FIG. 10B are plan views of a reusable diaper of the present invention in a flat-out state. FIG. 10A shows the outer surface of the diaper which faces away from user when worn, and FIG. 10B shows the inner surface of the diaper which faces toward the user's skin when worn. FIG. 10C shows the diaper of FIG. 10A and FIG. 10B when it is folded to form the arrangement adopted when the diaper is worn by a user.

### DETAILED DESCRIPTION

As set out above, the present invention provides a reusable article for retaining bodily fluids and solids.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The term “reusable article” as used herein refers to devices that are configured to retain bodily fluids and solids that a body excretes, for example, devices which are placed against or in proximity to the body of the wearer to collect and retain various exudates discharged from the body (e.g. urine and faeces). The reusable article is configured to be reused for more than one usage cycle of collecting and retaining bodily fluids and solids that a body exudates. Exemplary reusable articles for storing and holding bodily fluids and solids include diaper liners, sanitary pads, panty liners, and the like. The reusable articles may be used as a liquid retaining component in a garment, for example, garments that resemble underwear, such as diapers (e.g. training pants, pull-on pant-type diapers, refastenable diapers, pant-type diapers), incontinence briefs, or feminine hygiene undergarments, and the like.

The reusable article of the present invention comprises a liquid impermeable inner layer and a liquid impermeable outer layer. The liquid impermeable inner layer comprises a first surface and a second surface, wherein the first surface is configured to face towards the skin of a user when the reusable article is in use. The liquid impermeable outer layer and the liquid impermeable inner layer are attached to each other around their peripheries such that a liquid retention chamber is formed between the second surface of the liquid impermeable inner layer and the liquid impermeable outer layer. For example, the liquid impermeable outer layer and the second surface of the liquid impermeable inner layer may be attached to each other around their peripheries to form the liquid retention chamber. Typically, the liquid impermeable outer layer and the liquid impermeable inner layer are attached to each other around the entirety of their peripheries. Optionally, the liquid impermeable inner layer and/or the liquid impermeable outer layer comprise flange portions that extend outwardly from the point of attachment between the two layers. The flange portions may also be configured to fold inwards towards the first surface of the liquid impermeable inner layer, and may optionally be elasticated. For example, the flange portions may be formed or comprise yarns having elastic properties. The flange portions can help to form a barrier around the periphery of the reusable article to help direct any liquid through the holes of the liquid impermeable inner layer.

Preferably, the liquid impermeable inner layer and liquid impermeable outer layer are attached to each other such that any liquid retained within the liquid retention chamber is unable to leak from the liquid retention chamber at the point of attachment between the two layers. Typically, the liquid impermeable inner layer and liquid impermeable outer layer are attached to each other by stitching, by an adhesive (e.g. the adhesive may be an adhesive tape, liquid glue, or hotmelt powder glue), and/or by ultrasonic bonding. When the adhesive is an adhesive tape, the tape may be a single-sided, double-sided adhesive tape and said tape may have a single layer or multiple layers where said multiple layers may have one or more functions, such as barrier layers, elastic layers etc. When the adhesive is a liquid glue, the glue may be a hot melt glue, a liquid resin or combinations thereof (e.g. the adhesive may be a hot melt glue/liquid resin bonding by nozzle extrusion or liquid resin bonding by screen printing/template printing). In certain examples, the adhesive or ultrasonic bonding may be heat stable up to at least 190° C., which enables the reusable article to be washed and dried multiple times. Unless otherwise specified, when used herein, the term “heat stable” is intended to stipulate that there is no change in the physical state of the component in question after being subjected to the stated temperature for a period of time consistent with a machine drying cycle.

The liquid impermeable inner layer comprises a plurality of holes configured to allow a liquid to pass through the liquid impermeable inner layer to the liquid retention chamber. Thus, when the reusable article is in use, bodily fluids, such as urine or menstrual fluid discharged from the user, may pass through the liquid impermeable inner layer into the liquid retention chamber where the bodily fluids may be retained until removal from the reusable article.

The holes may be any size suitable to allow a liquid to pass through the holes in the absence of external forcing pressure. For example, the holes may have a diameter of about 0.5 cm to about 3 cm, for example, about 0.5 cm, about 1 cm, about 1.5 cm, about 2 cm, about 2.5 cm, or about 3 cm. The specific size of the holes is not particularly important provided that they are large enough to allow a liquid to pass through the liquid impermeable inner layer without forcing pressure.

For the avoidance of doubt, the term “about” as used herein can allow for a degree of variability in a value or range, for example, within 10%, within 5%, within 1%, within 0.5%, within 0.1%, within 0.05%, within 0.01%, within 0.005%, or within 0.001% of a stated value or of a stated limit of a range, and includes the exact stated value or range.

The liquid impermeable inner layer and liquid impermeable outer layer may each be formed from any suitable material that is impermeable to a liquid, in particular water in liquid form. The liquid impermeable inner layer and liquid impermeable outer layer may be formed from the same or different type of material. Typically, the material is a hydrophobic material. The material of the layers may allow water to escape when in gaseous form. Examples of suitable materials include, hydrophobic polymeric materials, durable water repellent (DWR) finishes on any fabric, hydrophobic finishes or films applied to any fabric, a silicon coating on any fabric, a polyurethane coating on any fabric, a hydrophobic spray treatment on any fabric, or a knitted/ woven liquid impermeable construction that uses hydrophobic yarns.

Optionally, the liquid impermeable outer layer may include one or more stitches that extend across a portion or an entire length of the liquid impermeable outer layer. For example, the one or more stitches may extend across the liquid impermeable outer layer from a first edge of the liquid impermeable outer layer to a second edge of the liquid impermeable outer layer. Typically, there may be two or more (e.g. 2, 3, 4, 5, 6 or more) stitches that each extend across the liquid impermeable outer layer. The one or more stitches may be any type of stitch suitable to reduce stretching of the liquid impermeable outer layer when the liquid retention chamber contains bodily fluids from a user. The one or more stitches may be one or more straight and/or zig-zag stitches that extend across the liquid impermeable outer layer.

In certain examples, the first surface of the liquid impermeable inner layer may be formed from an outer hydrophobic material, and the second surface of the liquid impermeable inner layer may be formed from an inner hydrophobic material. The outer hydrophobic material and the inner hydrophobic material may be formed from the same or different type of material, both being materials that are impermeable to a liquid, in particular water in liquid form.

The liquid retention chamber of the reusable article may house a wicking layer, the wicking layer being positioned adjacent to the plurality of holes in the liquid impermeable inner layer. The wicking layer has a first surface and a second surface. The first surface of the wicking layer faces the second surface of the liquid impermeable inner layer and the second surface of the wicking layer faces the liquid retention chamber. Typically, the first surface of the wicking layer is attached to the second surface of the liquid impermeable inner layer and covers the plurality of holes. The wicking layer acts to wick liquid away from the holes of the liquid impermeable inner layer and direct it to the liquid retention chamber. The wicking layer also acts as a barrier to solid exudates, thus filtering the bodily fluids from any solid exudates, the solids being retained on the first surface of the wicking layer. Because bodily fluids pass through the liquid impermeable inner layer and wicking layer into the liquid retention chamber, exposure of the user's skin to the bodily fluid can be reduced. This can increase comfort for the user once the reusable article is soiled and/or reduce any skin irritation that may be caused by extended contact of the user's skin to the bodily fluids. By only permitting bodily fluids to pass into the liquid retention chamber, the reusable article can be easier to clean after soiling by a user.

For the avoidance of doubt, the term “attached” as used herein refers to elements that are connected or coupled together by stitching, bonding, fastening, adhering etc. Many methods for attaching elements together are known in the art, and include, for example, adhesive bonding (e.g. laminate bonding), pressure bonding, thermal bonding, ultrasonic bonding, mechanical fastening, and the like. The means by which the elements are attached is preferably heat stable up to at least 190° C., which may enable the reusable article to be washed and dried multiple times.

In certain examples, the wicking layer and liquid impermeable inner layer may be laminated together. For example, the two layers may be laminated together by a laminating adhesive between the wicking layer and the second surface of the liquid impermeable inner layer. Suitable laminating adhesives include, but are not limited to, polyamide adhesives, polyester adhesives, polyurethane adhesives, and mixtures of two or more adhesives thereof. By laminating the two layers together it is possible to simplify the assembly process of the reusable article of the present invention.

The wicking layer may be formed from any material that is suitable for wicking fluid away from the one or more holes of the liquid impermeable inner layer. Typically, the wicking layer may be formed from fibres, or yarns made with fibres, where said fibres and yarns are selected from one or more of the group consisting of polyamide, polyester, polyolefin, polyurethane, polyacrylonitrile, natural cellulose, regenerated cellulose, regenerated cellulose derivatives (i.e. cellulose acetate and cellulose triacetates), natural protein and regenerated protein. The wicking layer may be produced using technologies such as knitting (warp knitting such as raschel Tricot, weft knitting such as circular or flat), weaving, non-woven methods (blow spinning, staple nonwoven, spun laid, air-laid, needle punched, thermal bonded, hydro-entangled, chemical bonded and so forth), electro-spinning, force-spinning etc. Additionally, the wicking layer may include one or more of coatings, treatment encapsulation or entrapment, which would enhance its liquid and moisture management functionality, such as rate of wicking, wicking capacity, rate of spreading and distribution, one-way liquid transport etc.

As an example, the wicking layer may comprise a material that is 51% cotton and one or both surfaces (i.e. the first and second surfaces) of the wicking layer may be treated with a hydrophilic composition or material (e.g. polyethylene oxide, polyvinyl alcohol, polyacrylamide, poly acrylic acid, polyvinyl pyrrolidone, hydrophilic silicones, or hydrophilic polyurethanes) and/or a hydrophobic composition or material (e.g. silicones, polyfluoroalkylacrylates, polyacrylates, polyurethanes, or waxes) to create a net hydrophilic gradient across the wicking layer.

The wicking layer may be configured to wick fluid away from the plurality of holes in the liquid impermeable inner layer and towards the liquid retention chamber. For example, the first surface of the wicking layer, which faces the second surface of the liquid impermeable inner layer, may be more hydrophilic (i.e. less hydrophobic) than the second surface of the wicking layer, which faces the liquid retention chamber. For example, the first surface may be treated with a hydrophilic material and/or the second surface may be treated with a hydrophobic material. This may result in a combination of a “pulling” force generated by the hydrophilic properties of the surface in contact with the liquid impermeable inner layer and a “pushing” force generated by the hydrophobic properties of the second surface facing the liquid retention chamber. This arrangement may wick any moisture or liquid through the wicking layer and away from the second surface of the liquid impermeable inner layer. The hydrophilic and hydrophobic compositions may be applied to the wicking layer using any conventional method known to the skilled person.

The liquid retention chamber of the reusable article may further house a liquid absorbent material. Typically, the liquid absorbent material is disposed between the second surface of the wicking layer and the liquid impermeable layer.

Additionally, or alternatively, the liquid retention chamber of the reusable article may further house a liquid collection vessel. Typically, the liquid collection vessel is disposed between the second surface of the wicking layer and the liquid impermeable outer layer. The liquid collection vessel may be attached, for example by bonding (e.g. adhesive bonding or ultrasonic) or stitching, to the second surface of the wicking layer. The surface of the liquid collection vessel adjacent to the wicking layer is configured to allow one-way fluid flow into the liquid retention chamber. For example, the liquid collection vessel may comprise one or more liquid non-return values. Typically, the one or more liquid non-return values may be formed from a polymer material such as silicone. The one or more liquid non-return valves may be positioned adjacent to the second surface of the wicking layer, such that when liquid passes through the holes of the liquid impermeable inner layer and through the wicking layer, the liquid enters the liquid collection vessel where it is retained until removal by a user. The liquid collection vessel may also be equipped with an outlet valve, such as a tap that a user can open to remove any liquid retained within the liquid collection vessel. The walls of the liquid collection vessel may be formed from any suitable material that is impermeable to a liquid, in particular water in liquid form. Suitable materials include those described above with respect to the liquid impermeable inner layer and liquid impermeable outer layer.

The liquid impermeable outer layer may comprise an opening that allows access to the liquid retention chamber. For example, a surface of the liquid impermeable outer layer may comprise an opening that allows access into the liquid retention chamber, the opening being reversibly sealed, for example by a reversible and water-tight seal (e.g. a water-tight zip fastener, or a water-tight hook and loop fastener). Such an opening allows access to the liquid retention chamber to allow removal of any bodily fluids retained in the chamber, and then allows for the chamber to be sealed for further use. For example, the opening may allow removal of a soiled liquid absorbent material for cleaning or replacement with a non-soiled liquid absorbent material. Or, for example, the opening may allow removal of the liquid collection vessel and/or access to an outlet valve of the liquid collection vessel.

The liquid absorbent material may be formed from any material that is suitable for absorbing and retaining a liquid. Suitable liquid absorbent materials are known in the art, and include, for example, cotton, a cotton blend, foam, a synthetic material, absorbent polymeric foam, a nanotechnology-based or -produced material, or any other moisture-absorbent material. The liquid absorbent material may have a weight of from about 50 g/m² to about 500g/m², such as from about 180 g/m² to about 300g/m². For example, the liquid absorbent material may be made from an 80:20 blend of polyester: nylon fabric with a microfiber double terry knit. Other suitable liquid absorbent materials include polypropylene or any cellulose-based fabric and their blends including cotton, bamboo etc.

In certain examples, the liquid absorbent material may be a 100% polyester double terry fabric. Such a material is approximately 90% air and so allows for a higher liquid absorbent capacity, as moisture fills up the air gaps of the polyester terry fabric without significant expansion of the polyester fibres.

In certain other examples, the liquid absorbent material may be made from a blended fibre comprising two or more of superabsorbent polymer (SAP), hydrogel and polyester, or at least part of the liquid absorbent material may have been treated with SAP and/or hydrogel. In such examples, these materials may result in increased liquid absorbent capacity, with a reduced thickness and weight for the liquid absorbent material.

In certain examples, the liquid absorbent material is formed from one or more of a hollow fiber material, a terry cloth, a knitted material, a woven material, and a hydrogel.

The liquid absorbent material may be formed of at least one layer of liquid absorbent material. The at least one layer of liquid absorbent material may be folded to create void spaces within the liquid retention chamber. The folds may be organised folds (e.g. zig-zag folds or pipe folds) or the folds may be disorganised (e.g. the liquid absorbent material or unit may be “scrunched-up” within the chamber). The void spaces allow for the liquid absorbent material or unit to expand in volume within the liquid retention chamber as it absorbs liquid that passes through the holes into the liquid retention chamber.

In certain examples, the liquid absorbent material may be a liquid absorbent unit that comprises a liquid absorbent layer and a barrier layer. The liquid absorbent layer may be formed from a suitable material for absorbing and retaining a liquid, such as those described hereinabove as being suitable for the liquid absorbent material. The barrier layer may be formed from any suitable material that retains water in liquid form, such as those described hereinabove as being suitable for the liquid impermeable inner layer and liquid impermeable outer layer of the reusable article. The presence of a barrier layer in the liquid absorbent unit can enhance liquid retention by the liquid absorbent material.

The liquid absorbent material or liquid absorbent unit may comprise a water-sensitive mechanism such as a water-sensitive indicator, treatment, or dye that indicates when the liquid absorbing capacity of the liquid absorbent material has been reached. For example, the liquid absorbent material or unit may include a portion or layer that is dyed with a suitable water-sensitive dye. Suitable water sensitive dyes may include those that change colour when contacted with liquid, such as urine. For example, the liquid absorbent material or liquid absorbent unit may have been treated, and therefore comprise, a hydrochromic dye. Suitable hydrochromic dyes are known in the art and commercially available. A change in the dye (e.g. a change in colour) may be detected by opening the releasable opening in the liquid impermeable outer layer. Additionally, or alternatively, the liquid impermeable outer layer may comprise a window configured such that the water-sensitive dye is visible through the liquid impermeable outer layer. The window may be sealed by a suitably transparent polymer material, for example, but not limited to, a polycarbonate film, a polyethylene terephthalate film, a polyvinyl chloride film, or a polyethylene film. Additionally, or alternatively, the liquid retention chamber may comprise a detector for fullness detection, for example, a dye detector that detects a change (e.g. a colour change) in the dye, which indicates that the liquid retaining capacity of the liquid absorbent material or unit has been reached. The dye detector may be configured to alert the user when the capacity of the liquid absorbent material or unit has been reached.

Additionally, or alternatively, the liquid retention chamber may comprise a smart sensor for real-time monitoring of the contents of the liquid retention chamber. For example, a smart sensor that detects and alerts a user when the maximum capacity of bodily fluids in the liquid retention chamber has been reached.

The reusable article may further comprise a liquid impermeable overlayer that extends over a portion (e.g. a central or a substantially central portion) or substantially all of the first surface of the liquid impermeable inner layer. For example, the liquid impermeable overlayer may extend over at least about 10%, at least about 40%, at least about 50%, at least about 60%, least about 70%, or at least about 80%, at least about 90%, at least about 99%, or about 100%, of the first surface of the liquid impermeable inner layer.

The liquid impermeable overlayer comprises a plurality of recesses. At least one of the plurality of recesses exposes one or more of the holes of the liquid impermeable inner layer. For example, one recess may expose one or more holes of the liquid impermeable inner layer.

The liquid impermeable overlayer may be an integral part of the liquid impermeable inner layer. Thus, the liquid impermeable inner layer may comprise the liquid impermeable overlayer, wherein the liquid impermeable overlayer forms part of the first surface of the liquid impermeable inner layer. For example, the liquid impermeable overlayer may be in the form of an embossed surface of the liquid impermeable inner layer that forms a plurality of recesses on the first surface of the liquid impermeable inner layer.

Alternatively, the liquid impermeable overlayer may be a separate layer and be disposed over, and attached to, the first surface of the liquid impermeable inner layer. When the liquid impermeable overlayer is a separate layer, it has a first surface and a second surface, the first surface being configured to face towards the skin of a user when in use, and the second surface being disposed over, and attached to, the first surface of the liquid impermeable inner layer.

At least one of the recesses exposes one or more of the holes of the liquid impermeable inner layer. Typically, one recess exposes one hole. In certain examples, each of the recesses present in the liquid impermeable overlayer expose one or more holes of the liquid impermeable inner layer. In certain other examples, one set of the recesses expose one or more holes of the liquid impermeable inner layer, and another set of recesses comprises a base that is substantially liquid impermeable. For example, when the liquid impermeable overlayer is a separate layer, one set of the recesses may expose one or more holes of the liquid impermeable inner layer, and another set of the recesses may only expose portions of the first surface of the liquid impermeable inner layer.

The recesses may have a well-like structure, a trough-like structure, a valley-like structure, or a vent-like structure. Typically, the recesses are well-like structures that are substantially cone, step pyramid, cuboid, cylindrical, circular based frustoconical or rectangle based frustoconical shaped.

The recesses of the liquid impermeable inner layer may all have the same shape, or they may have different shapes, for example, the recesses may each independently have a shape selected from substantially cone, step pyramid, cuboid, cylindrical, circular based frustoconical or rectangle based frustoconical shaped.

The recesses may be shaped such that the diameter of the recesses increases in an outwardly direction from the liquid impermeable inner layer. Thus, the widest portion of the recesses are positioned adjacent to the skin of a user, when the reusable article is in use. When the recesses have this arrangement, they can act as funnels to direct bodily fluids from the user to the holes of the liquid impermeable inner layer and into the liquid retention chamber.

The recesses of the liquid impermeable overlayer may act as receptacles for solid exudates, such as faeces, discharged from a user. Retention of solid exudates within the recesses can reduce exposure of the user's skin to the solid exudates, which can increase comfort of the user once the reusable article is soiled and/or reduce any skin irritation that may be caused by the solid exudates.

The liquid impermeable overlayer may be constructed from a hydrophobic foam (e.g. a polyurethane foam), structures formed from polyurethane, polyethylene, polystyrene and rubber-based structures; and silicon structures (e.g. a silicone gel, silicone puff, a silicone extrusion structure, etc.). The liquid impermeable overlayer may be formed by injection moulding using a suitable liquid impermeable polymer, for example a suitable thermoplastic or thermosetting polymer. Alternatively, the liquid impermeable overlayer may be fabric engineered to have recesses, for example, a fabric with a double jersey structure with tuck or miss stitches to create the recesses. Such a material can be prepared using techniques and equipment known in the art, such as a double/Jacquard knitting machine. Optionally, the liquid impermeable overlayer may comprise a hydrophobic finish/treated fabric, for example, a hydrophobic finish/treated fabric on an outer surface of the liquid impermeable overlayer and/or a surface within the recesses. The presence of a hydrophobic finish/treated fabric that provides a hydrophobic surface on an outer surface of the liquid impermeable overlayer and/or within the recesses facilitates the removal of any liquid or solid exudates retained within the recesses or otherwise disposed on a surface of the liquid impermeable overlayer.

The reusable article may be a component for a reusable garment, such as a diaper, a training pant, a pull-on pant-type diaper, a refastenable diapers, a pant-type diaper, or an incontinence brief, or an undergarment. For example, the reusable article may be a diaper liner, a liner for an incontinence brief, a sanitary pad, or a panty liner. In such examples, reusable article may be fitted to a garment. The reusable article may be fitted to a garment after the garment has been manufactured, for example, fitted to a garment by a user. Or, for example, the reusable article may be incorporated into a garment during the manufacture of the garment to form an integral part of such a garment. That is to say that the reusable article as described herein may form part of a garment, whether integral or attachable/removable.

Thus, the present invention further provides a reusable garment comprising a reusable article of the present invention. The reusable garment may comprise a main fabric body configured to be worn by a user and a reusable article of the present invention attached to an interior surface of the fabric body and extending over at least an area subject to bodily exudates. Any garment that is intended to be in contact with a user's skin may be fitted with one or more reusable articles of the present invention. Typically, the reusable garment is a diaper (e.g. a training pant, a pull-on pant-type diaper, a refastenable diapers, a pant-type diaper), an incontinence brief, or feminine hygiene undergarment, or the like. The level of internal surface area coverage of the reusable article in the reusable garment can be readily determined by the skilled practitioner based upon the intended use and the desired level of comfort of the wearer.

The reusable garment may include a component that seals the garment to the body of a user when in use. This may be achieved using any conventional means, such as elastic, the use of silicone to create a seal (including flocked silicone), ruching, etc. For example, the garment may include a nano-grip elastic portion at a leg-opening region of the reusable garment. A nano grip elastic is a type of elastic to grip against the skin to provide a barrier effect. For example, it may be used to create a stand-up barrier around the leg-openings to reduce leakage from the reusable garment. The nano-grip elastic can provide better fit and avoid loose opening around the leg.

In examples where the reusable garment is a diaper (e.g. a training pant, a pull-on pant-type diaper, a refastenable diapers, a pant-type diaper), an incontinence brief, or feminine hygiene undergarment, or the like, the garment typically has the form of an underpant. The garment may include a fabric body having a waist opening and a pair of leg openings defining a crotch area there between that covers some or all of the genital area of a user. The garment will have an internal surface that contacts a user's skin when in use. The garment will also have an external surface, where some or all of said external surface is not in direct contact with the user's skin when in use. The garment comprises a reusable article of the present invention that is attached to an interior surface of the fabric body of the garment. The reusable article may be attached to the garment with stitching, or it may be attached using adhesive, adhesive tape or some other form of attachment.

In certain examples, the reusable article is reversibly attached to the garment, for example using a reversible attachment means such as, but not limited to, buttons and button holes, zip fastener, press studs and sockets, hooks and eyes, and hook and loop fasteners (e.g. Velcro®). Reversible attachment is particular useful when the reusable article is to be fitted to a garment by a user (e.g. when the reusable article is a diaper liner, a liner for an incontinence brief, a sanitary pad, or a panty liner, or the like, which is to be fitted to a garment by a user).

In certain examples, the reusable garment is a diaper, for example a training pant, a pull-on pant-type diaper, a refastenable diapers, a pant-type diaper. Typically, when the garment is a diaper, it is a diaper for an infant. In examples where the garment is a diaper, the diaper may have a substantially hourglass configuration when open. It may include expandable leg openings and a waistband fitted with reversible attachment means such as, but not limited to, buttons and button holes, zip fastener, press studs and sockets, hooks and eyes, and hook and loop fasteners (e.g. Velcro®), such reversible attachment means arranged to allow for adjustment of the size of the waistband.

In certain examples, the reusable article, and the reusable garment comprising the reusable article, can withstand multiple machine wash cycles and/or tumble-dry cycles. For example, the reusable article, and the reusable garment comprising the reusable article, may be capable of withstanding at least 30 (e.g. a minimum of 50 or 100) machine wash and tumble dry cycles without change in overall appearance, integrity of the components and liquid management parameters as discussed herein. For example, the reusable article, and the reusable garment, may be reusable up to a minimum of 30 (e.g. a minimum of 50 or 100) wash and dry cycles. The material selection and unique construction ensures that the reusable article, and reusable garment, is washable without compromising on its function. Furthermore the integrity of the reusable article may be maintained for a minimum of 30 (e.g. a minimum of 50 or 100) wash and dry cycles. For example, the individual elements of the reusable article specifically mentioned herein are chosen such that they remain chemically, thermally and mechanically stable throughout the intended lifetime of the product while undergoing up to 30 (e.g. up to 50 or up to 100) wash and dry cycles.

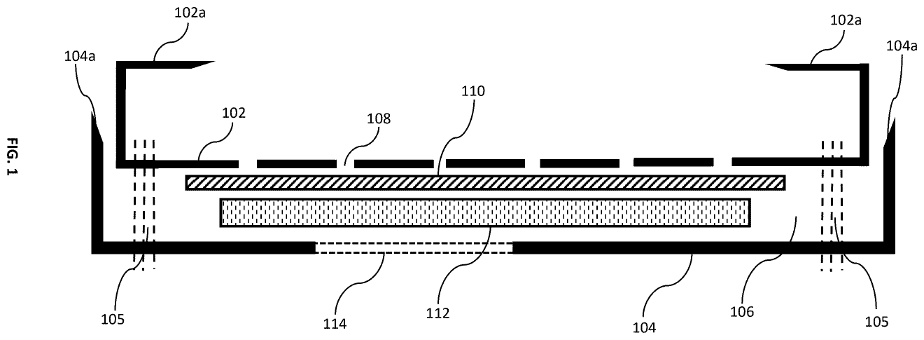

With reference to the drawing figures, FIG. 1 shows a partially exploded cross-sectional view of an exemplary reusable article 100 of the present invention. The article has a liquid impermeable inner layer 102 having a first surface and a second surface, the first surface is configured to face towards the skin of a user when the reusable article is in use. The reusable article also has a liquid impermeable outer layer 104, having an outer surface and an inner surface. The second surface of the liquid impermeable inner layer 102 is disposed over the liquid impermeable outer layer 104. The liquid impermeable outer layer 104 and the liquid impermeable inner layer 102 are attached to each other about their peripheries, thereby forming a liquid retention chamber 106 between the second surface of the liquid impermeable inner layer 102 and the inner surface of the liquid impermeable outer layer 104. The liquid impermeable inner layer 102 comprises a plurality of holes 108 that are configured to permit a liquid to pass through the layer into the liquid retention chamber 106. The liquid impermeable inner layer 102 and the liquid impermeable outer layer 104 are attached to each other by stitching 105, although bonding (e.g. adhesive or ultrasonic bonding) may additionally or alternatively be used. The liquid impermeable inner layer 102 and the liquid impermeable outer layer 104 have flange portions 102a, 104a that extend outwardly from the point of attachment between the two layers (i.e. from the point that the liquid impermeable outer layer 104 and the liquid impermeable inner layer 102 are attached to each other, for example by stitching). The flange portions provide a barrier around the periphery of the reusable article to help direct any bodily fluids from the user through the holes 108 of the liquid impermeable inner layer 102. The reusable article also has a wicking layer 110 positioned adjacent to the plurality of holes 108 in the liquid impermeable inner layer 102, such that each hole 108 exposes a portion of the wicking layer 110. Specifically, the wicking layer 110 is disposed between the second surface of the liquid impermeable inner layer 102 and the inner surface of the liquid impermeable outer layer 104, thus being positioned within the liquid retention chamber 106. The wicking layer 110 and second surface of the liquid impermeable inner layer 102 may be laminated together. The wicking layer 110 is formed from a material that is suitable for wicking fluid away from the one or more holes 108 of the liquid impermeable inner layer 102. The wicking layer 110 has a first surface that faces the second surface of the liquid impermeable inner layer and a second surface that faces the liquid retention chamber 106. The liquid retention chamber 106 may optionally house a liquid absorbent material 112 in the form of a layer of a material that is suitable for absorbing and retaining a liquid. When the liquid absorbent material 112 has reached its liquid absorbance capacity, the liquid absorbent material 112 may occupy substantially all of the internal volume of the liquid retention chamber 106. The liquid absorbent material 112 when in a dry form may be arranged within the liquid retention chamber 106 such that it has one or more folds that form void spaces within the liquid retention chamber 106. The folds may be organised folds (e.g. zig-zag folds or pipe folds) or the folds may be disorganised (e.g. the liquid absorbent material may be “scrunched-up” within the chamber). The void spaces allow the liquid absorbent material 112 to expand in volume within the liquid retention chamber 106 as it absorbs liquids passing through the holes 108 into the liquid retention chamber 106. When in use, bodily fluids from a user, such as urine, pass through the holes 108 and into the liquid retention chamber 106. The wicking layer 110 acts to wick the fluid away from the holes 108 and direct it to the liquid absorbent material 112 housed within the liquid retention chamber 106 where the fluid is retained until the liquid absorbent material 112 is removed via opening 114 in the liquid impermeable inner layer 104.

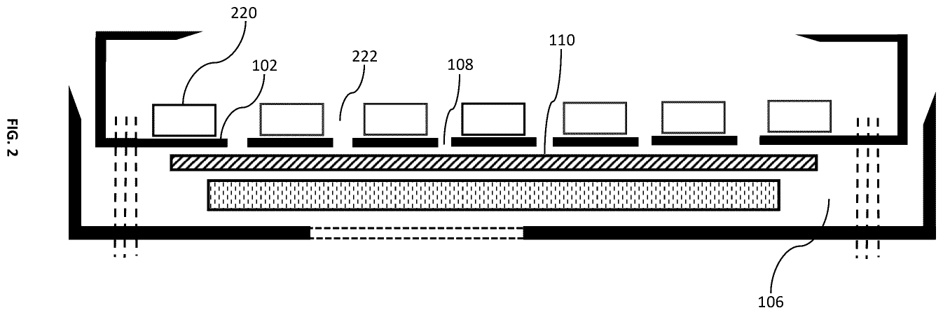

FIG. 2 shows a partially exploded cross-sectional view of the exemplary reusable article 100 shown in FIG. 1 additionally comprising a liquid impermeable overlayer 220 that extends over the first surface of the liquid impermeable inner layer 102. The liquid impermeable overlayer 220 is disposed over and attached to the first surface of the liquid impermeable inner layer 102, for example by bonding (e.g. adhesive or ultrasonic bonding). The liquid impermeable overlayer 220 comprises a plurality of recesses 222 in the form of well-like structures. The recesses 222 each expose a hole 108 in the liquid impermeable inner layer 102, and a portion of the wicking layer 110 which is attached to the second surface of the liquid impermeable inner layer 102. The recesses 222 may each have a cylindrical shape. Other shapes are also possible, for example, the recesses may each have a cone, a step pyramid, a cuboid, circular based frustoconical, or a rectangle based frustoconical shape. The recesses 222 may each have a hydrophobic surface that facilitates removal of bodily fluids and/or solids disposed on the walls of the recesses after the reusable article has been soiled by a user. When in use, bodily fluids from a user, such as urine, enter the recesses 222, which act as funnels that direct the fluids through the holes 108 and into the liquid retention chamber 106. The recesses 222 can act as receptacles for retaining solids, such as faeces, excreted from the user.

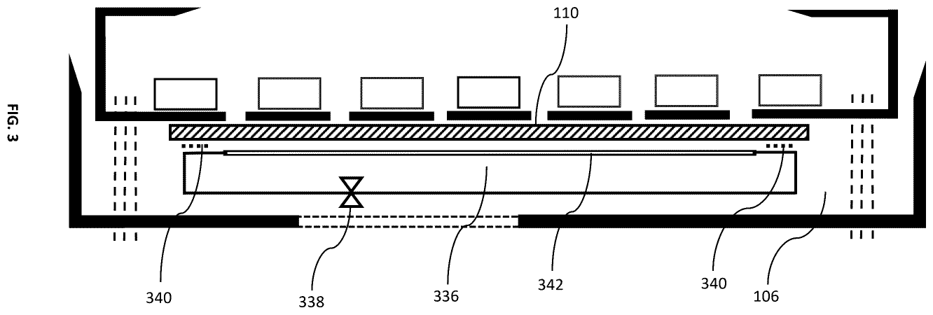

FIG. 3 shows a partially exploded cross-sectional view of the exemplary reusable article 100, wherein the liquid retention chamber 106 houses a liquid collection vessel 336 rather than a liquid absorbent material. The liquid collection vessel 336 has an outlet valve 338 in form of a tap, which allows a user to remove liquids retained in the liquid collection vessel 336. The liquid collection vessel 336 has a top surface, which is in contact with the second surface of the wicking layer 110. The liquid collection vessel 336 and wicking layer 110 are attached to each other around their peripheries by bonding 340 (e.g. adhesive tape). The top surface of the liquid collection vessel comprises a liquid non-return valve 342, which allows liquid to enter the liquid collection vessel 336 where it is retained until a user removes the liquid via outlet valve 338.

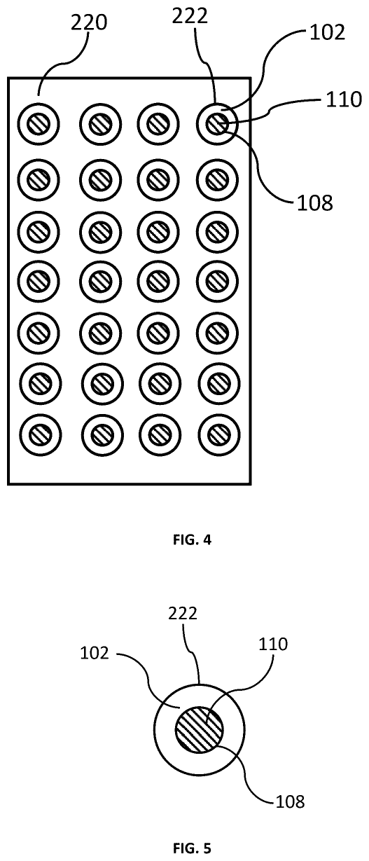

FIG. 4 shows an isolated top view of the exemplary reusable article 100 shown in FIGS. 1, 2 and 3. This figure does not show the flange portions and any liquid impermeable inner layer that may extend past the peripheries of the liquid impermeable overlayer. The recesses 222 in the liquid impermeable overlayer 220 each expose a hole 108 in the liquid impermeable inner layer 102 and a portion of the wicking layer 110. FIG. 5 shows a magnified view of a single recess 222, exposing a hole 108, a portion of the first surface of the liquid impermeable inner layer 102, and a portion of the wicking layer 110, which is exposed by the hole 108.

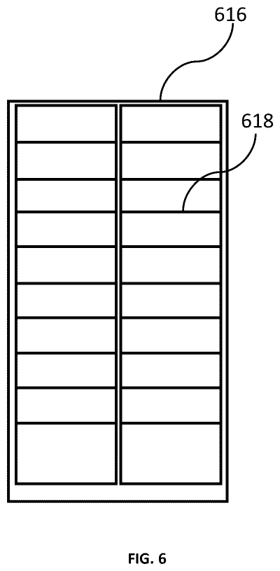

FIG. 6 shows a top view of a liquid absorbent unit 616 which may be contained within the liquid retention chamber of the reusable article of the present invention. The liquid absorbent unit 616 has folds 618, which create void spaces within the liquid absorbent unit. The liquid absorbent unit 616 may comprises a barrier layer and a liquid absorbent layer. The two layers may be attached to each other by adhesive (e.g. laminating adhesive), bonding or stitching.

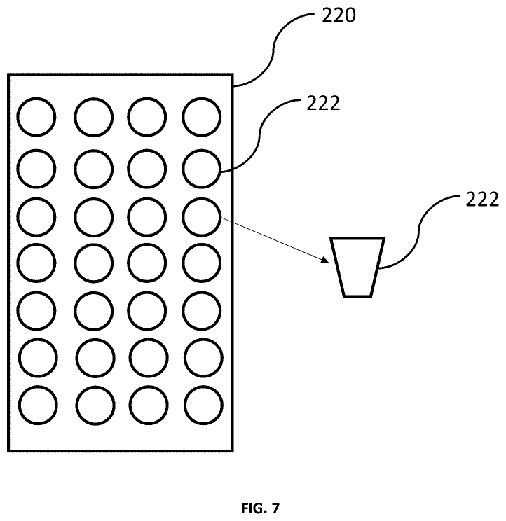

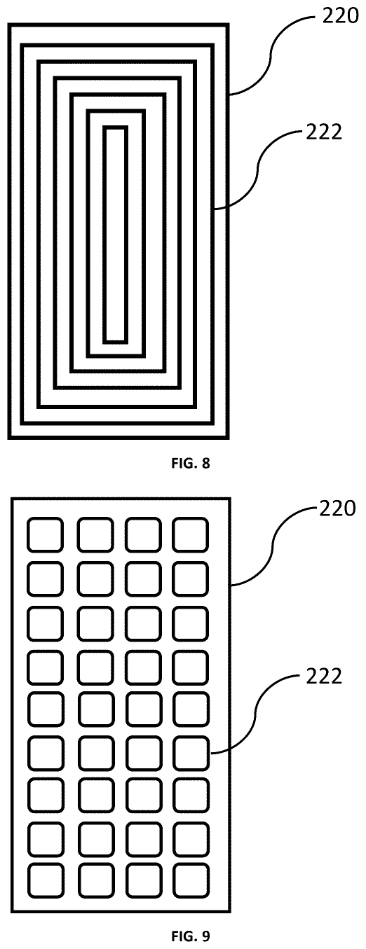

FIG. 7 shows a top view of an alternative liquid impermeable overlayer 220, together with an isolated cross sectional image of one of the recess 222 which has a frustoconical shape. The narrow base of the recess 222 is configured to expose a hole of the liquid impermeable inner layer. FIG. 8 shows a further alternative liquid impermeable overlayer 220 wherein the recesses 222 of the liquid impermeable overlayer 220 are in the form of vents, and FIG. 9 shows a further alternative liquid impermeable overlayer 220 wherein the liquid impermeable overlayer 220 has a waffle-like structure, with each recess 222 having a rectangle based frustoconical shape.

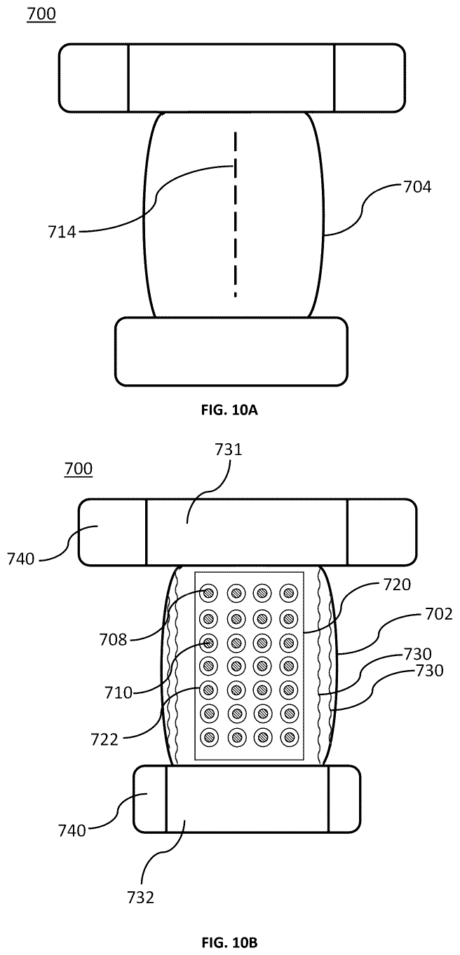

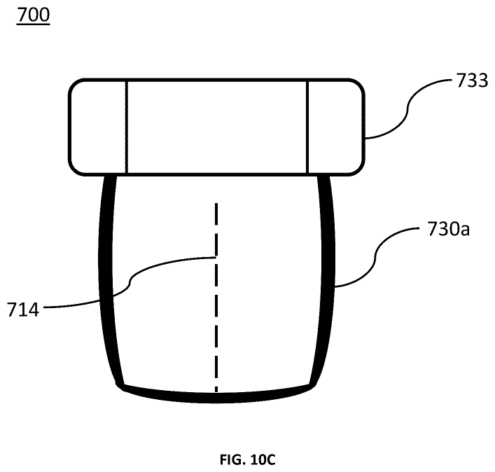

FIG. 10A and FIG. 10B are plan views of a diaper 700 of the present invention in a flat-out state. FIG. 10A shows the outer surface of the diaper which faces away from wearer during use, and FIG. 10B shows the inner surface of the diaper which faces toward the user's skin during use. The outer surface shown in FIG. 10A is formed by a liquid impermeable outer layer 704, and the inner surface of the diaper shown in FIG. 10B is formed from a liquid impermeable inner layer 702 and a liquid impermeable overlayer 720. The liquid impermeable inner layer 702 has holes 708. The liquid impermeable overlayer 720 is disposed over a central portion of the liquid impermeable inner layer 702, recesses 722 of the liquid impermeable overlayer 720 each exposing a hole 708, and a portion of a wicking layer 710. The liquid impermeable outer layer 704 and liquid impermeable inner layer 702 are attached to each other around the entirety of their peripheries by stitching, thereby forming a liquid retention chamber between the two layers. The liquid impermeable outer layer 704 has an opening 714 that allows access to the liquid retention chamber. The diaper has elasticized leg cuffs 730 formed from flange portions extending outwardly from the liquid impermeable outer layer 704 and liquid impermeable inner layer 702; elastic waistband regions 731, 732; and a fastening system 740.

FIG. 10C shows the diaper 700 when folded to form the arrangement adopted when the diaper is worn by a user, the elasticized legcuffs 730 forming expandable leg openings 730a and the elastic waistband regions 731, 732 forming an elasticated waistband 733 that would encompass the circumference of a user's waist when in use. The opening 714 allowing access to the liquid retention chamber.

The fastening system 740 typically maintains the first waistband region 731 and the second waist region 732 in a configuration that provides lateral tension about the circumference of the diaper 700 which holds the diaper 700 on the user when in use. The fastening system 740 may comprise a reversible attachment means such as, but not limited to, buttons and button holes, zip fastener, press-studs and sockets, hooks and eyes, and hook and loop fasteners (e.g. Velcro®), although any other known fastening means are generally acceptable. In an alternative example, opposing sides of the diaper may be seamed or welded together to form an underpant structure, which allows the diaper to be used as a pull-on type diaper, such as a training pant.

Various aspects of the present reusable article may also be understood from the following clauses:
1. A reusable component for storing and holding bodily fluids and solids, the reusable component comprising:
   a liquid impermeable barrier layer, the liquid impermeable barrier layer having a first surface and a second surface, the first surface configured to face towards the skin of a user when in use;
   a liquid impermeable vessel comprising a liquid impermeable sealing layer that is attached to a periphery of the second surface of the liquid impermeable barrier layer to form a liquid impermeable seal between the layers and also define a liquid retention chamber, wherein
   the liquid impermeable barrier layer further comprises a plurality of holes to allow a liquid to pass into the liquid retention chamber.
2. The reusable component according to Clause 1 or 2, wherein the liquid impermeable barrier layer further comprises a liquid impermeable overlayer that extends from the first surface of the liquid impermeable barrier layer, where the a liquid impermeable overlayer comprises a plurality of recesses that expose the first surface of the liquid impermeable barrier layer, where at least some of the recesses also expose the plurality of holes
3. The reusable component according to Clause 2, wherein at least one of the plurality of holes is exposed in each of the plurality of recesses.
4. The reusable component according to Clause 2 or Clause 3, wherein the plurality of recesses are formed from one or more of a plurality of well-like structures, a plurality of trough-like structures and a plurality of valley-like structures,
   optionally wherein the plurality of recesses are formed from a plurality of well-like structures,
   more optionally wherein the well-like structures have a shape selected from the group consisting of a cone, a step pyramid, cuboid, circular based frustoconical and rectangle based frustoconical.
5. The reusable component according to any one of the preceding clauses, wherein the liquid retention chamber houses a wicking layer configured to be positioned adjacent to the plurality of holes in the liquid impermeable barrier layer, the wicking layer suitable for wicking fluid away from the plurality of holes.
6. The reusable component according to any one of the preceding clauses, wherein the liquid retention chamber further houses a liquid absorbent material.
7. The reusable component according to Clause 6 as dependent on Clause 5, wherein the wicking layer is configured to wick fluid from the plurality of holes to the liquid absorbent material.
8. The reusable component according to Clause 6 or 7, wherein the liquid absorbent material is provided in the form of at least one layer of liquid absorbent material.
9. The reusable component according to Clause 8, wherein at least one layer of liquid absorbent material is folded to create void spaces.
10. The reusable component according to any one of Clauses 6 to 9, wherein the liquid absorbent material is provided as a liquid absorbent unit comprising a liquid absorbent material layer and a liquid impermeable barrier layer.
11. The reusable component according to Clause 10, wherein the liquid absorbent unit comprises an absorbent material layer.
12. The reusable component according to any one of Clauses 6 to 11, wherein the liquid absorbent material is formed from one or more of a hollow fiber material, a terry cloth, a knitted material, a woven material, and a hydrogel.
13. The reusable component according to any one of the preceding clauses, wherein the liquid impermeable vessel is openable to allow access to the liquid retention chamber,
   optionally wherein:
      (A) the bottom wall or at least one side wall comprise a water-tight opening (e.g. a water-tight zipper) for allowing opening of the liquid retention chamber; or
      (B) the at least one side wall is configured to releasable connect to the liquid impermeable barrier layer.
14. The reusable component according to any one of the preceding clauses, further comprising a smart sensor for real-time monitoring of the liquid retention chamber.
15. The reusable component according to Clause 10 or 11, or according to any one of Clauses 12 to 14 as dependent on Clause 10 or 11, wherein the absorbent material further comprises a dye, and the liquid retention chamber comprises a dye detector for fullness detection.
16. The reusable component according to any one of the preceding clauses, wherein:
the first surface of the liquid impermeable barrier layer is formed from an outer hydrophobic material, and the second surface of the liquid impermeable barrier layer is formed from an inner hydrophobic material;
   the liquid retention chamber houses a liquid absorbent material; and
   the liquid retention chamber houses a wicking layer configured to be positioned adjacent to the plurality of holes in the liquid impermeable barrier layer, the wicking layer suitable for wicking fluid from the plurality of holes to the liquid absorbent material.
17. The reusable component according to Clause 16, wherein the liquid impermeable vessel is openable to allow access to the liquid retention chamber,
   optionally wherein:
      (A) the bottom wall or at least one side wall comprise a water-tight opening (e.g. a water-tight zipper) for allowing opening of the liquid retention chamber; or
      (B) the at least one side wall is configured to releasable connect to the liquid impermeable barrier layer.
18. The reusable component according to any one of the preceding clauses, wherein the holes are configured to allow liquid to pass into the liquid retention chamber but to prevent solids from passing into the liquid retention chamber.
19. A reusable garment comprising the reusable component according to any one of the preceding clauses.
20. The reusable garment according to Clause 19, wherein the reusable garment is a reusable undergarment.
21. The reusable undergarment according to Clause 19 or 20, wherein the reusable garment is machine washable,
   optionally wherein the reusable garment is able to withstand at least 30 machine wash and tumble dry cycles.

Although the disclosure herein refers to certain example embodiments, those embodiments are present by way of example and not be way of limitation. The intent of the foregoing detailed description is to be construed to cover all modification, alternatives, and equivalents of the embodiments as may fall within the scope of the present invention as defined by the claims.

1. A reusable article for retaining bodily fluids and solids, the reusable article comprising:
a liquid impermeable inner layer having a first surface and a second surface, the first surface configured to face towards the skin of a user when in use;
a liquid impermeable overlayer that extends over a portion or all of the first surface of the liquid impermeable inner layer; and
a liquid impermeable outer layer;
the liquid impermeable outer layer and the liquid impermeable inner layer being attached to each other around their peripheries, thereby forming a liquid retention chamber between the second surface of the liquid impermeable inner layer and the liquid impermeable outer layer,
the liquid impermeable outer layer comprising a reversibly sealed opening that allows access to the liquid retention chamber.
the liquid impermeable inner layer comprising a plurality of holes to allow a liquid to pass through the liquid impermeable inner layer into the liquid retention chamber,
wherein the liquid retention chamber houses a wicking layer, the wicking layer positioned adjacent to the plurality of holes, and
the liquid impermeable overlayer comprises a plurality of recesses, wherein at least one of the recesses exposes one or more of the plurality of holes.

2. (canceled)

3. The reusable article according to claim 1, wherein the plurality of recesses are formed from one or more of a plurality of well-like structures, a plurality of trough-like structures, a plurality of vent-like structures, and a plurality of valley-like structures.

4. (canceled)

5. The reusable article according to claim 1, wherein the liquid retention chamber houses a liquid absorbent material.

6. The reusable article according to claim 5, wherein the wicking layer is configured to wick fluid from the plurality of holes to the liquid absorbent material.

7. The reusable article according to claim 5, wherein the liquid absorbent material is provided in the form of at least one layer of liquid absorbent material.

8. The reusable article according to claim 7, wherein at least one layer of liquid absorbent material is folded to create void spaces.

9. The reusable article according to claim 5, wherein the liquid absorbent material is provided as a liquid absorbent unit comprising a liquid absorbent layer and a barrier layer.

10. The reusable article according to claim 5, wherein the liquid absorbent material is formed from one or more of a hollow fiber material, a terry cloth, a knitted material, a woven material, and a hydrogel.

11. The reusable article according to any claim 1, wherein the liquid retention chamber houses a liquid collection vessel comprising walls formed from a liquid impermeable material,
the liquid collection vessel comprising one or more liquid non-return values positioned adjacent to the wicking layer, such that when liquid passes through the holes of the liquid impermeable inner layer and through the wicking layer, the liquid enters the liquid retention vessel where it is retained until removal by a user, and wherein the liquid collection vessel further comprising an outlet valve configured to allow removal of any liquid retained within the liquid collection vessel.

12. The reusable article according to claim 1, wherein the reversibly sealed opening is sealed by a reversible and water-tight seal.

13. The reusable article according to claim 1, further comprising a smart sensor for real-time monitoring of the contents of the liquid retention chamber.

14. The reusable article according to claim 5, wherein the absorbent material further comprises a water-sensitive indicator, treatment, or dye.

15. The reusable article according to claim 1, wherein:
the first surface of the liquid impermeable inner layer is formed from an outer hydrophobic material, and the second surface of the liquid impermeable inner layer is formed from an inner hydrophobic material;
the liquid retention chamber houses a liquid absorbent material; and
the liquid retention chamber houses a wicking layer configured to be positioned adjacent to the plurality of holes in the liquid impermeable inner layer, the wicking layer suitable for wicking fluid from the plurality of holes to the liquid absorbent material.

16. The reusable article of claim 1, wherein the reusable article is a diaper liner, a liner for an incontinence brief, a sanitary pad, or a panty liner.

17. A reusable garment comprising the reusable article of claim 1.

18. The reusable garment of claim 17, wherein the reusable garment is a reusable undergarment.

19. The reusable garment according to claim 17, wherein the reusable garment is machine washable.

20. The reusable article according to claim 12, wherein the reversible and water-tight seal is a water-tight zip fastener or a water-tight hook and loop fastener.

21. The reusable garment of claim 18, wherein the reusable garment is a diaper.

22. The reusable garment of claim 21, wherein the diaper is a training pant, a pull-on pant-type diaper, a refastenable diaper, or a pant-type diaper.
